(19) United States
(12) Patent Application Publication
(10) Pub. No.: US 2017/0128627 A1
(43) Pub. Date: May 11, 2017

(54) POROUS COMPOSITE FIBROUS SCAFFOLD FOR BONE TISSUE REGENERATION

(71) Applicants: Manitha B. NAIR, Kochi (IN); Deepthy Menon, Kochi (IN); Shantikumar Nair, Kochi (IN)

(72) Inventors: Manitha B. NAIR, Kochi (IN); Deepthy Menon, Kochi (IN); Shantikumar Nair, Kochi (IN)

(73) Assignee: Amrita Vishwa Vidyapeetham, Kochi (IN)

(21) Appl. No.: 15/341,866

(22) Filed: Nov. 2, 2016

(30) Foreign Application Priority Data
Nov. 2, 2015 (IN) 5919/CHE/2015

(51) Int. Cl. A61L 27/46; A61L 27/56

(52) U.S. Cl. CPC A61L 27/46; A61L 27/56; A61L 2430/02

(57) ABSTRACT

Aporous composite fibrous scaffold for repair or regeneration of bone is disclosed. The scaffold comprises a first biopolymer forming a porous matrix and a second biopolymer forming fiber reinforcement. The biocompatible scaffold is configured to maintain balance between porosity and mechanical strength which could aid cellular infiltration and bone tissue regeneration. A method of preparing the porous composite scaffold is also disclosed.

### CROSS REFERENCE TO RELATED APPLICATIONS

The present invention claims priority to Indian Patent Application no. 5919/CHE/2015 titled POROUS COMPOSITE FIBROUS SCAFFOLD FOR BONE TISSUE REGENERATION filed on Nov. 2, 2015.

### FIELD OF THE INVENTION

The present invention is related to a porous composite fibrous scaffold for repair or regeneration of bone. More specifically, the invention relates to a biocompatible scaffold that could aid cellular infiltration and bone tissue regeneration. Moreover, the present invention also relates to a method of preparing the porous composite scaffold.

### DESCRIPTION OF THE RELATED ART

Treatment of bone loss in situations like trauma, osteonecrosis and tumours demands the need of an ideal bone graft for repair and regeneration. Although autograft and allograft are used in current clinical treatment modalities, limitations such as donor site morbidity for autografts and immunological response and risk of disease transmission for allograft, necessitate looking for other alternatives for management of bone defects.

The synthetic bone graft, which mimics the mineral composition of bone such as hydroxyapatite (HA), has fulfilled the properties of a bone substitute. However, hydroxyapatite (HA) is resistant to degradation in vivo, which occurs at a rate of 1-2% per year and is highly brittle, making it unsuitable for load bearing applications. Some of the approaches mentioned in the prior art are as follows,

U.S. Pat. No. 7,879,093 discloses an artificial bone composite structure consists of a fibrous matrix with a fiber and a hydroxyapatite particle. The particles are dispersed within the fibrous matrix and aligned along the axes of the fibers. WO2013152265 discloses a silk microfiber-reinforced scaffolds to design the high strength composites used for bone graft applications.

WO2014160019 discloses a scaffold comprising an aligned electrospun fiber having adjacent major peaks with about 180° apart from each other in fast Fourier transform analysis. US20060199876 discloses a porous bioceramic matrix of continuous phase and a biocompatible polymer of continuous or discontinuous phase.

However, conventional electrospun micro/nano fiber reinforced hydroxyapatite-polymer scaffold create dense mats of fibers and the alignment of fibers was restricted to one or two planes. Thus, there is a need for development of porous polymer scaffolds reinforced with electrospun micro/nano fibers aligned throughout the matrix for bone regeneration. This may resist the fracture in a better manner than alignment in a single plane. In addition, the fiber incorporation should not affect the overall porosity and biocompatibility of the composite scaffold.

### SUMMARY OF THE INVENTION

In various embodiments a porous composite fibrous scaffold for repair or regeneration of bone is disclosed. The composite scaffold comprises one or more bioceramics blended with a first biopolymer forming a porous matrix and a second biopolymer forming fiber reinforcement. The one or more bioceramics form 10% to 90% by weight of the scaffold, the first biopolymer from 10% to 70% by weight of the scaffold, and the second biopolymer from 1% to 50% by weight of the scaffold.

In some embodiments the scaffold exhibits flexural strength of at least 5 MPa and flexural modulus of at least 250 MPa. In some embodiments the pore size of the scaffold is in the range 10-800 μm. In some embodiments the fiber reinforcement forms a three-dimensional structure with 50-95% porosity by volume of the scaffold. In some embodiments the fibers are in the form of continuous yarn of diameter ranging from 100-1000 μm, with individual fiber diameters of 100-1000 nm. In some embodiments the fibers are in the form of segmented yarn. In some embodiments the fibers are in the form of fluff, having individual fiber diameter ranging from 100-1000 nm.

In some embodiments the one or more bioceramics in the scaffold comprise hydroxyapatite, tricalcium phosphate, bioglass, apatite-wollastonite glass ceramic, or calcium phosphate. In some embodiments the first or the second biopolymer comprises a single polymer. In some embodiments the first or the second biopolymer comprises blend of polymers. In some embodiments the first polymer comprises one or more of gelatin, collagen, elastin, fibrin, agarose, chitin, chitosan, carboxymethyl chitosan, alginate, pullulan, starch or silk. In some embodiments the second polymer comprises one or more of poly(lactic acid), poly(lactic-co-glycolic acid), poly(caprolactone), poly(hydroxy butyrate), poly(hydroxy butyrate valerate), poly(urethane), poly(vinyl alcohol), poly(vinyl pyrrolidone), or their copolymers. In some embodiments the second polymer comprises gelatin, collagen, elastin, fibrin, agarose, chitin, chitosan, carboxymethyl chitosan, alginate, pullulan, starch, or silk.

In some embodiments of the scaffold, the fibers are aligned in the matrix in a random arrangement in more than one plane. In some embodiments the fibers are aligned in the matrix in a parallel alignment in more than one plane. In some embodiments the fibers are aligned in the matrix circumferentially in more than one plane.

In some embodiments the hydroxyapatite is coated with one or more of silica, strontium, zinc, iron, manganese, magnesium or tin. In some embodiments the hydroxyapatite is doped with one or more of silica, strontium, iron, manganese or tin.

A method of preparing a porous composite fibrous scaffold in various embodiments is disclosed, comprising the steps of a). preparing a slurry of one or more bioceramics with a first biopolymer to form a matrix, b). preparing a fiber reinforcement of a second biopolymer to render it hydrophilic, c). dispersing the fibrous reinforcement in the matrix to form a composite, d). generating porosity within the composite by freeze-drying, and e). cross-linking the first biopolymer to produce the porous composite fibrous scaffold.

In some embodiments the method in step a), preparing the slurry includes coating or doping the bioceramics with one or more of silica, strontium, zinc, iron, manganese, magnesium or tin. In some embodiments of the method the pore size of the scaffold is configured by varying the ratio of the one or more bioceramics to the first biopolymer in step a). In some embodiments of the method in step a) the first biopolymer comprises one or more of gelatin, collagen, elastin, fibrin, agarose, chitin, chitosan, carboxymethyl chitosan, alginate, pullulan, starch, or silk.

In some embodiments the method in step b), the second biopolymer comprises one or more of poly(lactic acid), poly(lactic-co-glycolic acid), poly(caprolactone), poly(hydroxy butyrate), poly(hydroxy butyrate valerate), poly(urethane), poly(vinyl alcohol), poly(vinyl pyrrolidone), or their copolymers and preparing the fiber comprises treating the fibers with one of 1-ethyl-3-(3-dimethylaminopropyl) carbodiimide (EDC), dopamine, or a peptide including arginine-glycine-aspartate to render the fibers hydrophilic.

In various embodiments of the method in step b), preparing the fiber comprises blending the second polymer with one or more natural polymers selected from gelatin, collagen, elastin, fibrin, agarose, chitin, chitosan, carboxymethyl chitosan, alginate, pullulan, starch, or silk and cross linking to render the fiber hydrophilic.

In various embodiments of the method in step c), the mechanical strength and toughness of the scaffold are configured by varying one or more of weight percentage, length, alignment or form of the fibers in the fiber reinforcement. In some embodiments of the method in step d), the porosity is configured to constitute 50 to 95% of the volume of the scaffold and the pore size is configured to be in the range 10 to 800 μm. In some embodiments of the method the cross-linking in step e) comprises cross-linking of the first polymer using one of glutaraldehyde, formaldehyde, 1-ethyl-3-(3-dimethylaminopropyl) carbodiimide, genipin, transglutaminase, caffeic acid, tannic acid, calcium chloride, formic acid or dextran dialdehyde or thermal induction

Other advantages and aspects of this invention will become apparent upon making reference to the specification, claims, and drawings to follow.

### BRIEF DESCRIPTION OF THE DRAWINGS

The invention has other advantages and features which will be more readily apparent from the following detailed description of the invention and the appended claims, when taken in conjunction with the accompanying drawings, in which:

FIG. 1 is a schematic diagram of a porous composite fibrous scaffold for repair or regeneration of bone.

FIG. 2 represents a method of preparing a porous composite fibrous scaffold.

FIG. 3 shows a schematic representation of the development of continuous or cut electrospun yarn reinforced polymer-bioceramic composite scaffold.

FIGS. 4A-4B illustrate scanning electron micrographs of electrospun yarn reinforced gelatin-silica coated hydroxyapatite (HA) composite scaffold.

FIG. 5A illustrates porosity evaluation of composite scaffold without fiber reinforcement and FIG. 5B illustrates porosity evaluation of composite scaffold with fiber reinforcement.

FIG. 5C presents mercury porosimetry evaluation of the composite fibrous scaffold.

FIG. 6A presents flexural strength data of the composite fibrous scaffold, FIG. 6B represents toughness (work of fracture) data, FIG. 6C represents load vs. extension curves and FIG. 6D illustrates flexural modulus data of the composite scaffold with varying content of reinforcing fibers.

FIG. 7A is a confocal optical micrograph showing the viable mesenchymal stem cells on composite scaffold after 24 h without fiber reinforcement, and FIG. 7B is an optical micrograph of viable stem cells on 10% fiber reinforced scaffold. FIG. 7C and FIG. 7D are scanning electron micrographs corresponding to the optical images of FIG. 7A and 7B, respectively.

FIG. 8A illustrates alkaline phosphatase activity and FIG. 8B illustrates osteocalcin release of mesenchymal stem cells as function of time on composite scaffold with or without fibers in comparison to commercially available hydroxyapatite (HA).

FIG. 9A shows the creation of critical sized segmental defect in the femur of a rat model, stabilized with stainless steel plates and screws.

FIG. 9B shows composite fibrous scaffold implanted in the critical sized segmental defect shown in FIG. 9A.

FIGS. 9C-9D illustrate reference with unfilled defect.

FIGS. 9E-9F and FIGS. 9G-9H show osseous tissue integration in rat model after 4 months, scaffold without fiber reinforcement (FIGS. 9E-9F) and scaffold with 10% fiber reinforcement (FIGS. 9G-9H).

FIGS. 9I-9J illustrate reference with commercially available hydroxyapatite (HA) in rat model after 4 months.

FIG. 10A-FIG. 10H represent histology evaluation of defect region implanted with unfilled defect (FIGS. 10A-10B), and composite scaffold without fiber reinforcement (FIGS. 10C-10D) and scaffold with 10% fiber reinforcement (FIGS. 10E-10F) after 2 and 4 months in comparison to commercially available hydroxyapatite (HA) (FIGS. 10G-10H).

### DETAILED DESCRIPTION

While the invention has been disclosed with reference to certain embodiments, it will be understood by those skilled in the art that various changes may be made and equivalents may be substituted without departing from the scope of the invention. In addition, many modifications may be made to adapt to a particular situation or material to the teachings of the invention without departing from its scope.

Throughout the specification and claims, the following terms take the meanings explicitly associated herein unless the context clearly dictates otherwise. The meaning of “a”, “an”, and “the” include plural references. The meaning of “in” includes “in” and “on.” Referring to the drawings, like numbers indicate like parts throughout the views. Additionally, a reference to the singular includes a reference to the plural unless otherwise stated or inconsistent with the disclosure herein.

The proposed invention relating to a porous composite fibrous scaffold for repair or regeneration of bone is described in the following sections referring to the sequentially numbered figures. The above-mentioned objectives are achieved by developing a biocompatible scaffold having balance between porosity and mechanical strength which could aid cellular infiltration and bone regeneration.

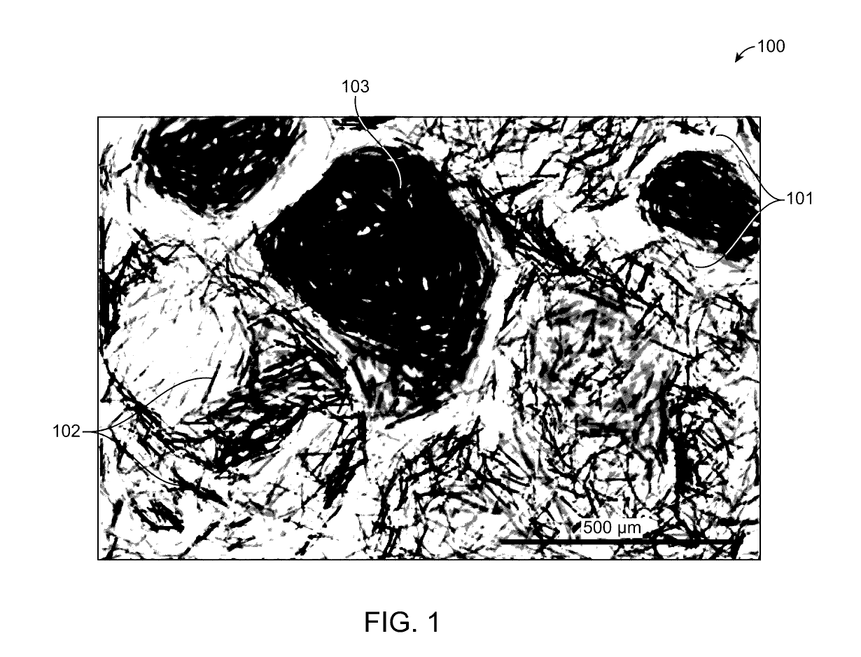

In one embodiment, a porous composite fibrous scaffold 100 for repair or regeneration or replacement of bone is disclosed, as shown in FIG. 1. The scaffold 100 comprises one or more bioceramics with a first biopolymer forming a matrix 101 with pores 103, and a second biopolymer forming fiber reinforcement 102 surrounding the pores 103. The scaffold 100 comprises bioceramics from 10% to 90% by weight of the scaffold, the first biopolymer from 10% to 70% by weight of the scaffold, and the second biopolymer from 1% to 50% by weight of the scaffold 100.

In one embodiment, the pore size of the scaffold 100 ranges from 10 μm to 800 μm and the fiber reinforcement 102 forms a three-dimensional structure within the matrix 101. In one embodiment the scaffold 100 displays porosity in the range of 50% to 95% by volume, which is optimal for bone regeneration. In one embodiment, the scaffold 100 exhibits flexural strength of at least 5 MPa and flexural modulus of at least 250 MPa, similar to cancellous or cortical bone. In one embodiment, the toughness of the scaffold may be at least 1 kJ/m². In various embodiments, the flexural modulus, flexural strength and toughness of the scaffold may be increased by optimally increasing the weight percentage of the fibers within the scaffold.

As illustrated in FIG. 1, the fiber reinforcement 102 in various embodiments is homogenously dispersed in the porous matrix 101 in a random manner or in a uniform parallel alignment across the matrix either unidirectionally, bidirectionally or angularly. Alternatively, the fiber reinforcement could be aligned circumferentially across the matrix in more than one plane. The three-dimensional structure of the scaffold 100 and arrangement of fiber reinforcement 102 are configured to enhance the mechanical properties of the scaffold 100, including strength and toughness. The structure of the scaffold is configured to provide an extracellular matrix (ECM) mimicking the natural micro environment, which is favourable for enhanced cellular adhesion, infiltration and bone formation. The interconnected porous structure is also configured to facilitate the ingrowth of cells and blood vessels, thereby improving tissue integration.

In some embodiments, the fibers are in the form of continuous yarn, segmented yarn or fluff. In some embodiments, the yarns or fluffy fibrous mass used as reinforcements in the scaffold may be produced by electrospinning. In some embodiments the mechanical strength and toughness of the scaffold may be adjusted by incorporating different weight percentage or different lengths or different alignment or different forms of reinforced fibers in the matrix. In one embodiment the fibrous yarns can be plied or heat stretched to enhance the mechanical strength.

In various embodiments, the first or the second biopolymer comprises a single polymer or a blend of polymers. In some embodiments, the first polymer is selected from one or more of gelatin, collagen, fibrin, elastin, silk, chitosan, agarose, chitin, carboxymethyl chitosan, alginate, pullulan, starch or silk. In some embodiments, the material of the second polymer making up the fibers comprises a suitable biocompatible synthetic polymer such as poly(lactic acid) (PLA), poly(glycolic acid) (PGA), poly(lactic-co-glycolic acid) (PLGA), poly(caprolactone) (PCL), polypropylene fumarate) (PPF), polyhydroxyalkanoate (PHA), poly(hydroxy butyrate) (PHB), poly(hydroxy butyrate valerate) (PHBV), poly(urethane) (PU), poly(vinyl alcohol) (PVA), poly(vinyl pyrrolidone) (PVP), or their copolymers, or blends thereof. In one embodiment, the material of the second polymer making up the fibers comprises a suitable natural polymer such as gelatin, collagen, elastin, fibrin, agarose, chitin, chitosan, carboxymethyl chitosan, alginate, pullulan, starch, silk or their blends. In some embodiments the strength and toughness of the composite are optimized by configuring the fiber reinforcement as a blend of more than one synthetic polymer or natural polymer or a blend of natural and synthetic fibers.

In some embodiments, one or more bioceramics may be incorporated within the matrix. The one or more bioceramics may include hydroxyapatite (HA), tricalcium phosphate, bioglass or apatite-wollastonite glass ceramic or calcium phosphate. In some embodiments the bioceramics may be provided as particulates coated or doped with one or more of elements such as silica, strontium, zinc, iron, manganese, magnesium or tin to enhance biologic response such as vascularization and bone regeneration in the body. In some embodiments, the scaffold may be incorporated with one or more growth factors such as platelet-derived growth factor (PDGF), transforming growth factor (TGF), platelet-derived angiogenesis factor (PDAF), vascular endothelial growth factor (VEGF), epidermal growth factor (EGF), insulin-like growth factor (IGF), fibronectin, bone morphogenetic protein (BMPs), fibroblast growth factor (FGF), platelet rich plasma or fibrin for enhancing vascularization and bone regeneration.

In some embodiments, the scaffold may comprise antibiotics from different classes such as aminoglycosides, carbapenems, cephalosporins, cyclic esters, quinolones, glycopeptides, glycylcycline, lipopeptides, macrolides and ketolides, monobactams, oxazolidinones, penicillins, polymyxins, rifamycins, amdinopenicillins, amphenicols, cephalosporins, lincosamides, penicillins, pleuromutilins, riminofenazines, streptogramins, tetracyclines, ansamycins, macrolides, aminoglycosides, chloramphenicol, lincosamides, cyclic polypeptides, glycopeptides or antibacterial agents including silver nanoparticles, gold nanoparticles, zinc oxide nanoparticles, magnesium oxide nanoparticles, copper oxide nanoparticles, titanium dioxide nanoparticles for treatment of a disease condition such as osteomyelitis. In some embodiments, the scaffold may be seeded with mesenchymal stem cells or pre-osteoblast cells or osteoblasts or endothelial cells or bone forming precursor cells and implanted as a tissue engineered construct. Further, genetically engineered mesenchymal stem cells can also be cultured on the scaffold for better integration with tissue.

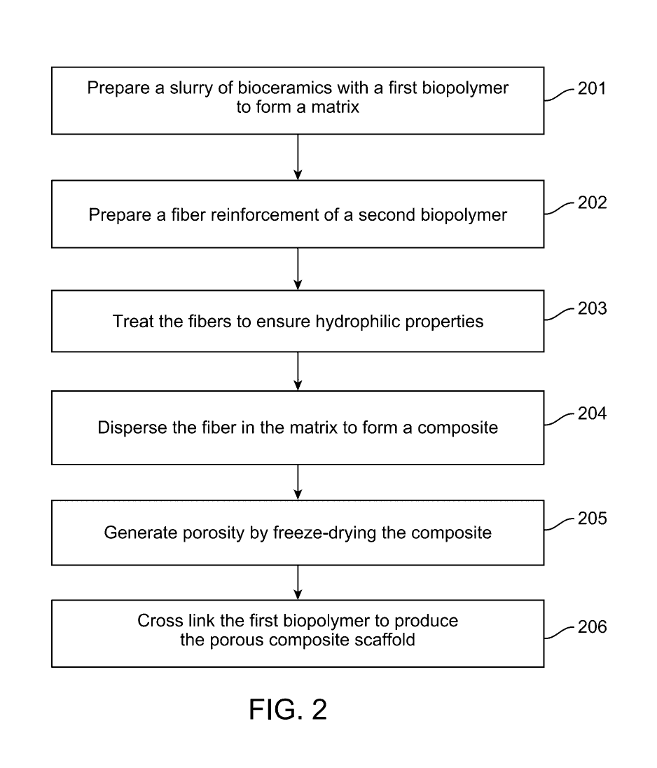

In one embodiment, a method of preparing a porous composite scaffold is disclosed, as shown in FIG. 2. Slurry of bioceramics with a first biopolymer is prepared to form a matrix in step 201 of the method. The bioceramic slurry with a first biopolymer is prepared in different weight percentages. In one embodiment, a fiber reinforcement of a second biopolymer is prepared in the form of continuous or segmented yarn or fluffy mass in step 202.

In one embodiment, the method includes treatment of the fibers to render them hydrophilic to provide improved interaction of the fiber to the matrix and to support attachment to mesenchymal stem cells (MSCs) or osteoblasts or endothelial cells or bone lining cells in step 203. In one embodiment, the method includes blending of synthetic and natural fibers and its cross linking for improved hydrophilicity in step 203. In one embodiment, the fiber in different length and weight percentage is homogenously dispersed in the matrix to form a composite in step 204. In one embodiment, the composite is then freeze dried to generate porosity in step 205; and followed by cross-linking the first biopolymer to produce the porous composite fibrous scaffold in step 206.

In various embodiments, the slurry may be prepared in step 201 using one or more bioceramics such as hydroxyapatite, tricalcium phosphate, bioglass or apatite-wollastonite glass ceramic or calcium phosphate singly or in combination. In various embodiments, prior to step 201 the bioceramics are coated or doped with one or more of silica, strontium, zinc, iron, manganese, magnesium or tin. In some embodiments of step 202, the mechanical strength and toughness of the scaffold may be adjusted by incorporating different weight percentage, different lengths, different alignment or different forms of the reinforcing fibers in the matrix.

In embodiments of step 203, the fibers are selected from the synthetic or natural polymers or blends developed by electrospinning. Preparing the fibers comprises treating the fibers with 1-ethyl-3-(3-dimethylaminopropyl) carbodiimide (EDC) or dopamine or RGD (arginine-glycine-aspartate) containing peptide to render them hydrophilic.

In various embodiments of step 204, the fiber reinforcement 102 (FIG. 1) is homogenously dispersed in porous matrix 101 (FIG. 1) in a random manner or in a uniform parallel alignment (unidirectional or bidirectional or angular) or circumferentially in more than one plane to form a three-dimensional structure of the scaffold 100. In one embodiment of step 205, the degradability and pore size of the scaffold are tailored to a particular application by varying the ratio of bioceramics to the first biopolymer. In one embodiment of step 205, the strength and toughness of the scaffold are optimized by use of fiber reinforcement comprising a blend of polymers of suitable mechanical characteristics. In one embodiment the scaffold displays porosity in the range of 50% to 95% by volume, which is optimal for bone regeneration. In one embodiment the cross-linking of the first polymer in step 206 is done by using a cross-linker such as gluteraldehyde or formaldehyde or 1-ethyl-3-(3-dimethylaminopropyl) carbodiimide (EDC) or genipin or transglutaminase or caffeic acid (CA) or tannic acid (TA)or calcium chloride orformic acid or dextran dialdehyde.

The scaffold in the various embodiments as disclosed above enhances cellular proliferation and osteogenic differentiation of mesenchymal stem cells and is therefore suitable for bone tissue regeneration, with application extending to areas of load bearing and non-load bearing requirements for both cortical and cancellous bony area. The bone-mimicking composition of the matrix material is an excellent osteoconductive material. The porosity induced in the material by freeze-drying technique enables cellular infiltration into the inner depths of the scaffold. The electrospun yarns or fluffy mass used as reinforcements in the scaffold could enhance the mechanical strength of the material. It could also provide an extracellular matrix (ECM) mimicking micro environment, which was favorable for enhanced cellular adhesion and bone formation. Mesenchymal stem cells are cultured on the scaffold, which shows good adhesion and viability on these scaffolds. The cells demonstrated alkaline phosphatase (ALP) activity and osteopontin expression, confirming the osteogenic differentiation of mesenchymal stem cells, as illustrated in the examples.

The invention is further explained in the following examples, which however, are not to be construed to limit the scope of the invention as delineated in the claims.

### EXAMPLES

### Example 1

### Development of Electrospun Yarn Reinforced Gelatin-silica Coated HA Matrix

Skeletal bones comprise mainly of collagen fibrils and carbonate substituted HA (35:65 ratio). These composites behave mechanically in a superior way to the individual components. Collagen fibrils constitute 90% of the organic phase of bone and are largely responsible for increasing fracture toughness and for providing resilience to bone. Calcium and phosphate in the form of HA is responsible for its stiffness and strength. In addition, trace elements like silica, strontium, zinc etc. have been shown to have positive effect on bone regeneration. Thus, a biomaterial engineered from such components is likely to behave similarly to native bone. In view of the above, a porous 3D scaffold made of gelatin-silica coated HA composite was developed, which was reinforced with electrospun fibrous yarns for bone tissue regeneration.

Electrospun micro/nano fibers of poly (L-lactic acid) (PLLA) were developed in the form of yarns and 3D fluffy mass. The hydrophobic nature of the PLLA fibers did not support mesenchymal stem cells (MSCs) and osteoblasts favourably. Therefore, PLLA yarns were treated with 1-ethyl-3-(3-dimethylaminopropyl) carbodiimide (EDC) to make it hydrophilic. Gelatin-HA slurry was prepared in different weight percentages to develop scaffolds with optimal pore size to mimic bone.

EDC treated PLLA yarns in different lengths and weight percentages were homogenously dispersed in the slurry, wherein orientation was random in more than one plane. It was then freeze dried to generate porosity followed by cross linking with gluteraldehyde. The degradability and pore size can be tailored to a particular application by varying the ratio between HA and gelatin. The mechanical strength, mainly toughness, can be adjusted by incorporating different weight percentages or different length or different forms of reinforced fibers. Mesenchymal stem cells were cultured on the scaffold, which showed good adhesion andviability on these scaffolds. The cells demonstrated alkaline phosphatase (ALP) activity and osteopontin expression, confirming the osteogenic differentiation of mesenchymal stem cells.

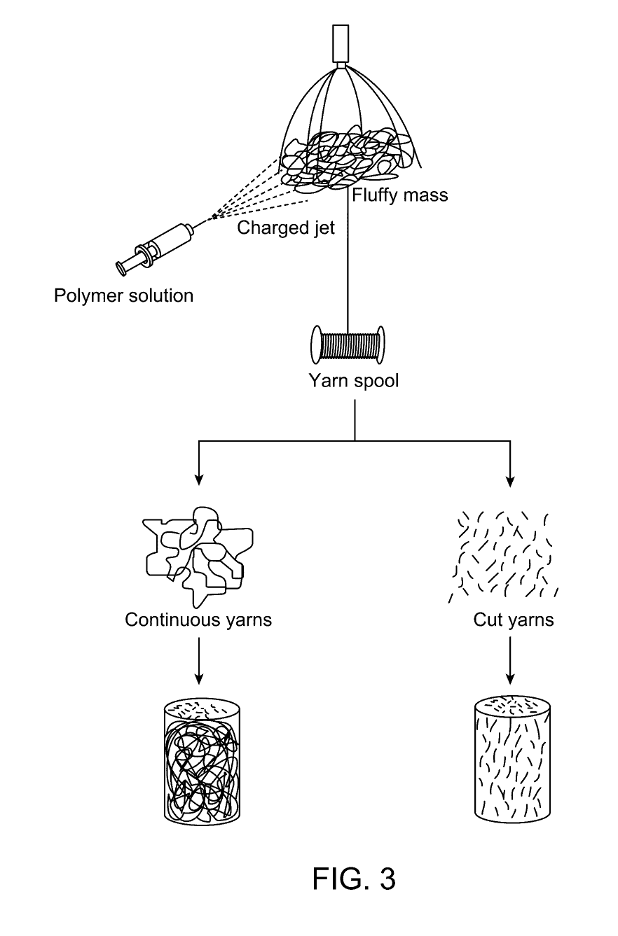

Schematic representation of the development of electrospun yarn reinforced polymer-bioceramics matrix is shown in FIG. 3. Continuous or segmented yarns or fluff can be loaded in biopolymer-bioceramics matrix in a random or aligned manner in more than one plane. Any suitable biocompatible natural or synthetic polymer can be utilized for making electrospun micro/nano fibers in connection with the present disclosure includingpoly(lactic acid) (PLA), poly(glycolic acid) (PGA), poly(lactic-co-glycolic acid) (PLGA), poly(caprolactone) (PCL), poly(propylene fumarate) (PPF), polyhydroxyalkanoate (PHA), poly(hydroxy butyrate) (PHB), poly(hydroxy butyrate valerate) (PHBV), poly(urethane) (PU), poly(vinyl alcohol) (PVA), poly(vinyl pyrrolidone) (PVP), and its copolymers, gelatin, collagen, elastin, fibrin, agarose, chitin, chitosan, carboxymethyl chitosan, alginate, pullulan, starch, silk or its blends. Likewise, instead of gelatin, the matrix can be made of collagen, fibrin, elastin, silk, chitosan agarose, chitin, chitosan, carboxymethyl chitosan, alginate, pullulan, starch or silk. Also, instead of HA, bioceramics like hydroxyapatite, tricalcium phosphate, bioglass or A-W glass ceramics or calcium phosphate or its combinations can be included in the matrix. The fibrous structure at the macro-scale can be in the form of yarns (continuous or segmented) or fluff. In addition, trace elements like one or more of one or more of silica, strontium, zinc, iron, manganese, magnesium or tin can be coated or doped on bioceramics to improve the characteristic of the matrix.

### Example 2

### Characteristics of the Composite Fibrous Scaffold

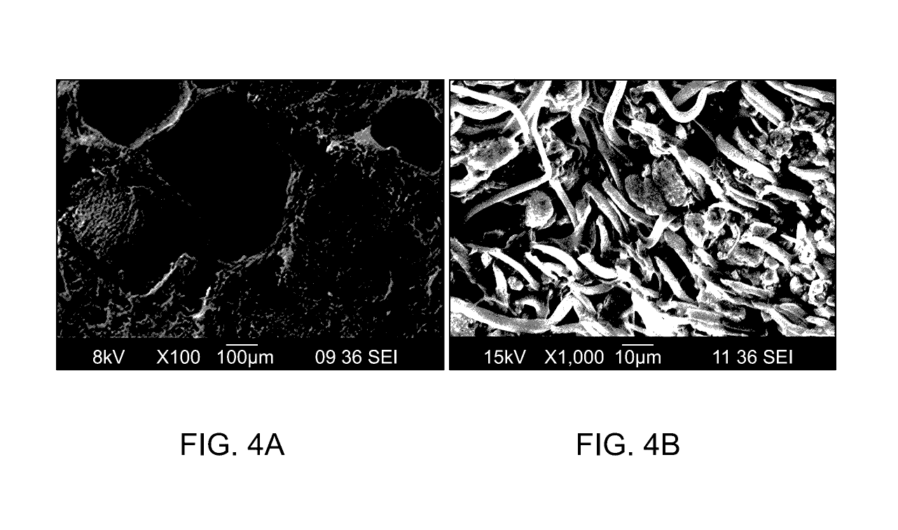

Scanning electron micrographs of electrospun yarns reinforced gelatin-HA composite scaffold are shown in FIGS. 4A-4B. The material was porous in nature with pores size in range of 50-500 μm. The fibrous yarns were well dispersed in gelatin-HA matrix in a random manner.

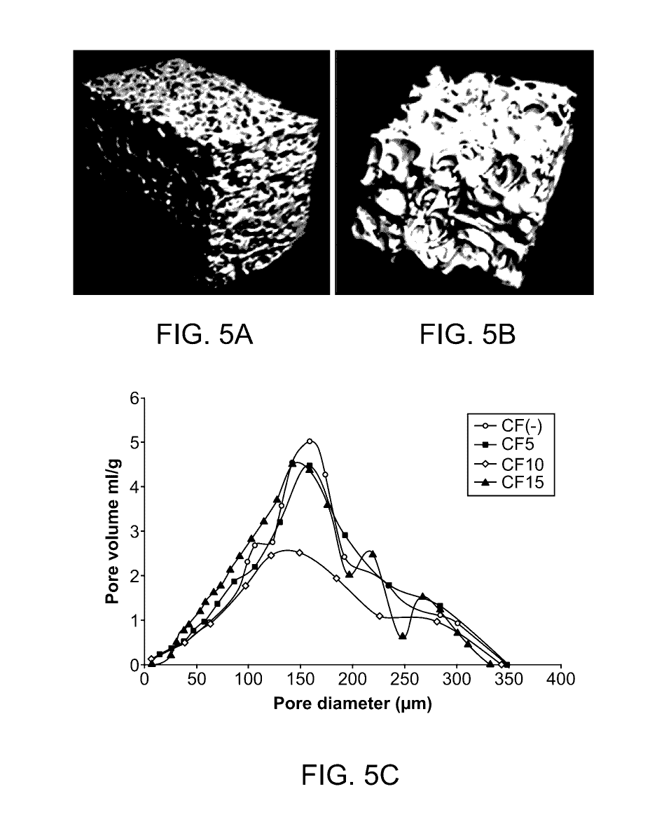

Porosity evaluation of composite scaffolds is shown in FIG. 5. Electrospun yarns were incorporated in three weight percentages (5, 10 and 15 wt %) to know whether fiber incorporation is having any impact on porosity. FIG. 5A and 5B show the micro CT 3D view of composite scaffolds (without fiber in FIG. 5A and with fibers in FIG. 5B). FIG. 5C shows the mercury porosimetric evaluation of scaffolds. Scaffold without fiber, scaffold with 5 wt % fibers, scaffold with 10 wt % fibers; scaffold with 15 wt % fibers are represented by CF(-),CFS, CF10, and CF15 respectively. All the scaffolds displayed porosity in the range of 60-75%, which is optimal for bone regeneration.

### Example 3

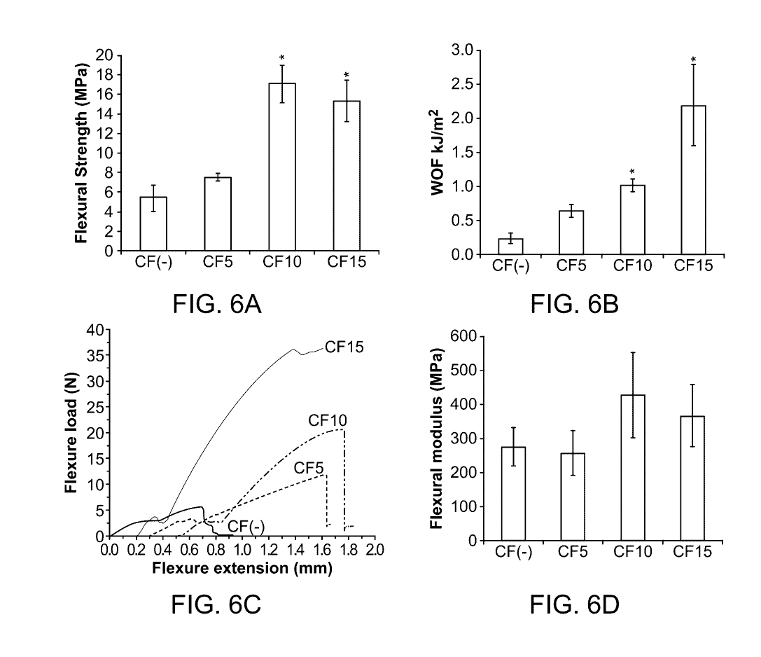

Mechanical strength analysis of composite scaffold with and without fibers is shown in FIG. 6A to FIG. 6D. FIG. 6A presents flexural strength data of the composite fibrous scaffold, FIG. 6B represents toughness (work of fracture) data, FIG. 6C represents load vs. extension curves and FIG. 6D illustrates flexural modulus data of the composite scaffold with varying content of reinforcing fibers. Electrospun yarns were incorporated in three weight percentages (5, 10 and 15 wt %). As observed in FIG. 6A-6D, the strength together with toughness was increased with increase in the weight percentage of fibers. Scaffold without fiber, scaffold with 5 wt % fibers, scaffold with 10 wt % fibers; scaffold with 15 wt % fibers are represented by CF(-), CF5, CF10, and CF15 respectively in the figures.

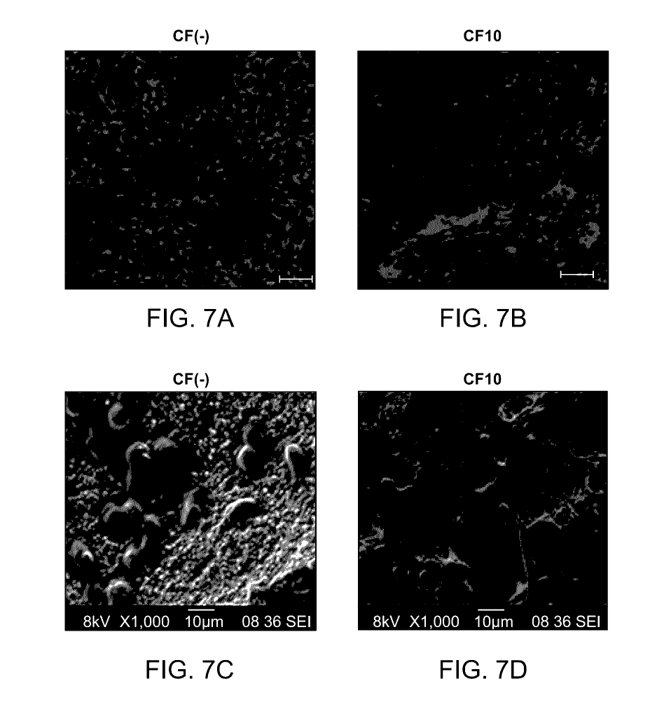

The adherence of mesenchymal stem cells on composite scaffolds after 24 h is shown in FIG. 7A-7D. The scaffold without fiber is represented by CF(-) and the scaffold with 10 wt % fibers is represented by CF10. FIG. 7A-7B show the confocal micrographs of the viable cells on scaffolds (calcein AM and ethidium bromide staining) without (FIG. 7A) and with 10% fiber reinforcement (FIG. 7B). FIG. 7C and 7D show the scanning electron micrographs of the adherence of cells on scaffolds. The cellular viability and adhesion was not altered due to the presence of fibers.

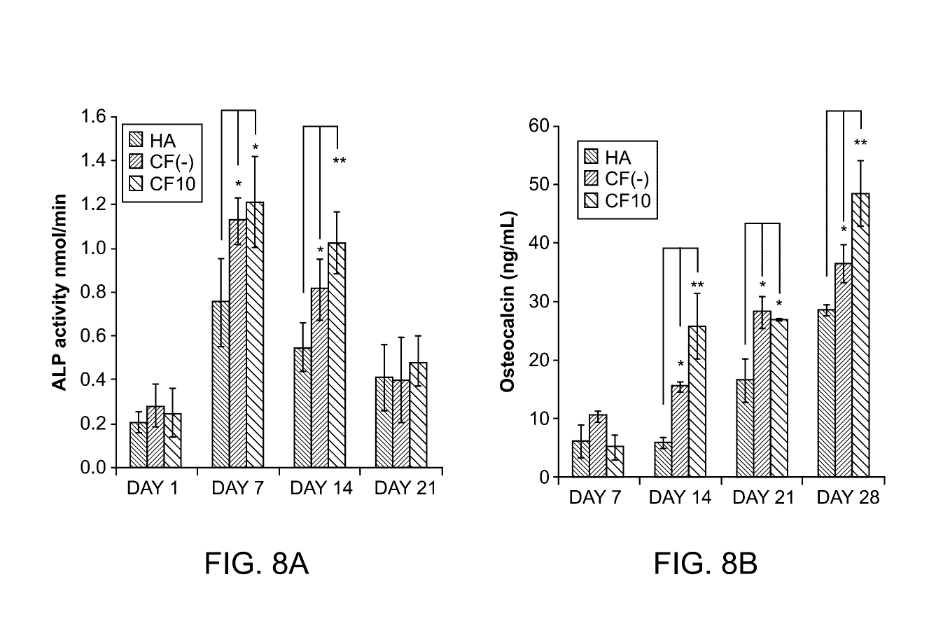

In FIG. 8A, alkaline phosphatase activity and in FIG. 8B, osteocalcin release of mesenchymal stem cells on the scaffold are shown, confirming osteogenic differentiation. Both the ALP activity and osteocalcin release was significantly high on composite scaffold with and without fibers when compared to commercially available HA.

### Example 4

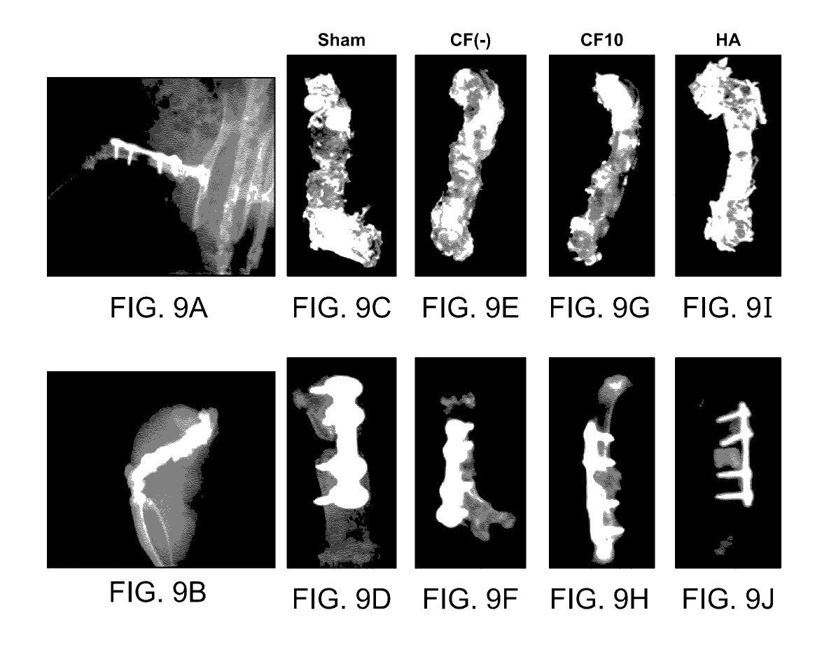

To determine the bone regeneration potential, the biomaterial was implanted in a critical sized segmental defect (5 mm defect) created in the femur of the rat model as shown in FIG. 9A-9[[F]]J. The defect was stabilized with stainless steel plates and screws as shown in FIG. 9A and implanted with the scaffold material in FIG. 9B. The upper portions (FIGS. 9C, 9E, 9G, and 9I) are optical photographs while the lower portions (FIGS. 9D, 9F, 9H, and 9J) represent X-ray images. Sham (FIGS. 9C-9D) represent the unfilled defect, CF(-) (FIGS. 9E-9F) show the scaffold without fiber, CF10 (FIGS. 9G-9H) show the scaffold with 10 wt % fibers and HA (FIGS. 91-9J) show the commercially available bio-graft HA. Left panel demonstrates the gross images and radiographs of the defect region after 4 months. There was no union in sham group or unfilled defect (FIGS. 9C-9D).

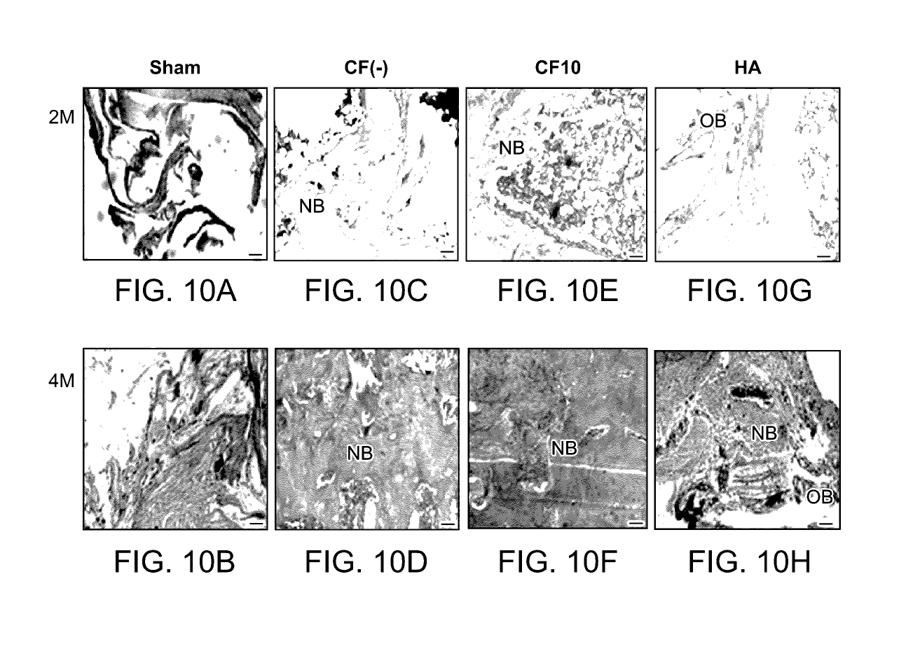

The material was retaining without any significant bone formation in commercially available HA group (FIGS. 9I-9J). But, the composite scaffold implants in FIGS. 9E-9F and 9G-9H exhibited an enhanced amount of bone formation and few remnants of materials were also seen. Histology evaluation of defect region implanted with biomaterials after 2 and 4 months is illustrated in FIGS. 10A-10H. The organized new bone formation was significantly enhanced in the defect implanted with composite scaffold in FIG. 10C-10D and 10E-10F. A few remnants of the scaffold materials were also seen.

While the above is a complete description of the preferred embodiments of the invention, various alternatives, modifications, and equivalents may be used. Therefore, the above description should not be taken as limiting the scope of the invention which is defined by the appended claims.

We claim:

1. A porous composite fibrous scaffold for repair or regeneration of bone, comprising:
one or more bioceramics blended with a first biopolymer forming a porous matrix; and
a second biopolymer forming a fiber reinforcement;
wherein the scaffold comprises the one or more bioceramics from 10% to 90% by weight of the scaffold, the first biopolymer from 10% to 70% by weight of the scaffold, and the second biopolymer from 1% to 50% by weight of the scaffold.

2. The scaffold of claim 1, wherein the scaffold exhibits flexural strength of at least 5 MPa and flexural modulus of at least 250 MPa.

3. The scaffold of claim 1, wherein the pore size of the scaffold is in the range 10-800 μm.

1. The scaffold of claim 1, wherein the fiber reinforcement forms a three-dimensional structure with 50-95% porosity by volume of the scaffold.

5. The scaffold of claim 1, wherein the fibers are in the form of continuous yarn of diameter ranging from 100-1000 μm, with individual fiber diameters of 100 1000 nm.

6. The scaffold of claim 1, wherein the fibers are in the form of segmented yarn.

7. The scaffold of claim 1, wherein the fibers are in the form of fluff, having individual fiber diameter ranging from 100-1000 nm.

8. The scaffold of claim 1, wherein the one or more bioceramics comprise hydroxyapatite, tricalcium phosphate, bioglass or apatite-wollastonite glass ceramic or calcium phosphate.

9. The scaffold of claim 1, wherein the first or the second biopolymer comprises a single polymer.

10. The scaffold of claim 1, wherein the first or the second biopolymer comprises a blend of polymers.

11. The scaffold of claim 1, wherein the first polymer comprises one or more of gelatin, collagen, elastin, fibrin, agarose, chitin, chitosan, carboxymethyl chitosan, alginate, pullulan, starch or silk.

12. The scaffold of claim 1, wherein the second polymer comprises one or more of poly(lactic acid), polylactic-co-glycolic acid), poly(caprolactone), poly(hydroxy butyrate), poly(hydroxy butyrate valerate), poly(urethane), polyvinyl alcohol), polyvinyl pyrrolidone), or their copolymers.

13. The scaffold of claim 1, wherein the second polymer comprises gelatin, collagen, elastin, fibrin, agarose, chitin, chitosan, carboxymethyl chitosan, alginate, pullulan, starch, or silk.

14. The scaffold of claim 1, wherein the fibers are aligned in the matrix in a random arrangement in more than one plane.

15. The scaffold of claim 1, wherein the fibers are aligned in the matrix in a parallel alignment in more than one plane.

16. The scaffold of claim 1, wherein the fibers are aligned in the matrix circumferentially in more than one plane.

17. The scaffold of claim 1, wherein the hydroxyapatite is coated with one or more of silica, strontium, zinc, iron, manganese, magnesium or tin.

18. The scaffold of claim 1, wherein the hydroxyapatite is doped with one or more of silica, strontium, iron, manganese or tin.

19. A method of preparing a porous composite scaffold comprising:
a. preparing a slurry of one or more bioceramics with a first biopolymer to form a matrix;
b. preparing a fiber reinforcement of a second biopolymer to render it hydrophilic;
c. dispersing the fiber reinforcement in the matrix to form a composite;
d. generating porosity within the composite by freeze-drying; and
e. cross-linking the first biopolymer to produce the porous composite scaffold,

20. The method of claim 19, wherein in step a), preparing the slurry comprises coating or doping the bioceramics with one or more of silica, strontium, zinc, iron, manganese, magnesium or tin.

21. The method of claim 19, wherein the pore size of the scaffold is configured by varying the ratio of the one or more bioceramics to the first biopolymer in step a).

22. The method of claim 19, wherein in step a) the first biopolymer comprises one or more of gelatin, collagen, elastin, fibrin, agarose, chitin, chitosan, carboxymethyl chitosan, alginate, pullulan, starch, or silk.

23. The method of claim 19, wherein in step b), the second biopolymer comprises one or more of poly(lactic acid), poly(lactic-co-glycolic acid), poly(caprolactone), poly(hydroxy butyrate), poly(hydroxy butyrate valerate), poly(urethane), polyvinyl alcohol), polyvinyl pyrrolidone), or their copolymers and preparing the fibrous structure comprises treating the fibers with one of 1-ethyl-3-(3-dimethylaminopropyl) carbodiimide (EDC), or dopamine, or a peptide including arginine-glycine-aspartate to render the fibers hydrophilic.

24. The method of claim 19, wherein in step b) preparing the fiber reinforcement comprises blending the second polymer with one or more natural polymers selected from gelatin, collagen, elastin, fibrin, agarose, chitin, chitosan, carboxymethyl chitosan, alginate, pullulan, starch, or silk and cross linking to render the fibers hydrophilic.

25. The method of claim 19, wherein in step c), the mechanical strength and toughness of the scaffold are configured by varying one or more of weight percentage, length, alignment or form of the fibers in the fiber reinforcement.

26. The method of claim 19, wherein in step d) the porosity is configured to constitute 50 to 95% of the volume of the scaffold and the pore size is configured to be in the range 10 to 800 μm.

27. The method of claim 19, wherein the cross-linking in step e) comprises cross-linking of the first polymer using one of glutaraldehyde, formaldehyde, 1-ethyl-3-(3-dimethylaminopropyl) carbodiimide, genipin, transglutaminase, caffeic acid, tannic acid, calcium chloride, formic acid or dextran dialdehyde.
